(19) United States
(12) Patent Application Publication
(10) Pub. No.: US 2026/0192131 A1
(43) Pub. Date: Jul. 9, 2026

(54) NEEDLE RF LASER TREATMENT SYSTEM AND METHOD FOR MANUFACTURING NEEDLE UNIT APPLIED THEREIN

(71) Applicant: TerasysD&C Inc., Namyangju-si (KR)

(72) Inventors: Dong Chae Lee, Seoul (KR); Kyu Tae KIM, Hanam-si (KR)

(21) Appl. No.: 19/335,987

(22) Filed: Sep. 22, 2025

(30) Foreign Application Priority Data
Jan. 9, 2025 (KR) 10-2025-0003687
Feb. 7, 2025 (KR) 10-2025-0016087

(51) Int. Cl. A61N 5/067; A61N 5/06

(52) U.S. Cl. CPC A61N 5/067; A61N 5/0601; A61N 5/0616; A61N 2005/0612; A61N 2005/063

(57) ABSTRACT

A needle RF Laser treatment system is disclosed. The needle RF Laser treatment system of the present disclosure has a needle unit including: a needle member provided in a main body and configured to penetrate the skin of a user; and a laser guide member coupled to the needle member and configured to supply laser to the needle member.

### CROSS REFERENCE TO RELATED APPLICATION

The present application claims priority to Korean Patent Applications Nos. 10-2025-0003687, filed Jan. 9, 2025 and 10-2025-0016087, filed Feb. 7, 2025, the entire contents of which are incorporated herein for all purposes by this reference.

### BACKGROUND

### Technical Field

The present disclosure relates to a treatment system, and more particularly, to a needle RF laser treatment system capable of maximizing treatment effects while minimizing damage to the skin of a user by using a laser and needles.

### Description of the Related Art

The skin treatment technology of the related art is classified into a non-invasive method using a laser and an invasive method using a needle member. The treatment method using a laser is mainly used to induce skin regeneration or to repair skin surface damage using thermal energy, and has an advantage that it is non-invasive. However, it is difficult to work effectively at a certain depth, and there is a limitation in that it is limited to superficial treatment.

The technology using a needle member has an advantage that it can promote collagen production or directly deliver drugs by creating micro-perforations in the skin. However, this method has a disadvantage in that it causes direct damage to the skin, so it results in pain and a long recovery time.

Recently, research has been conducted to combine laser and needle technologies in order to simultaneously take advantage of both technologies. For example, a technology of delivering laser energy through a needle member to efficiently deliver the energy to deep layers of the skin while reducing epidermal damage has attracted attention. However, these existing technologies show limitations in energy delivery efficiency and uniformity of treatment areas.

The above description is the background for helping understand the present disclosure and does not means the related art well known in the art.

### SUMMARY

The present disclosure has been made in an effort to solve the problems of the relate art described above, and is specifically intended to solve the following objectives.

First, to provide a structure capable of delivering laser energy more efficiently so that the energy can be uniformly delivered to deep layers of the skin.

Second, to expand the range of treatment areas and shorten the treatment time by distributing laser to a plurality of needle members through a laser guide member.

Third, to propose a measure capable of maximizing therapeutic effects while minimizing damage to the skin of a user.

Fourth, to provide higher energy efficiency and user convenience than those of skin treatment devices based on a single technology.

According to an aspect of the present disclosure, there may be provided needle RF laser treatment system having a needle unit including: a needle member provided in a main body and configured to penetrate the skin of a user; and a laser guide member coupled to the needle member and configured to supply laser to the needle member.

The needle member may include a plurality needle members, and the laser guide member may be coupled to each of the plurality of needle members to supply laser to each of the needle members.

The needle member may include: a first region having a first end protruding outward from the needle unit, penetrating the skin of a user, and disposed parallel to a propagation direction of laser; a second region connected to a second end of the first region and obliquely disposed inside the needle unit; and a third region disposed inside the needle unit, having a first end connected to the second region 242, and having a second end connected to a laser supply cable configured to supply laser.

The needle unit may include: a needle housing coupled to the main body; and a needle base coupled to the needle housing and configured to support the needle member exposed outside the needle housing.

The needle unit may further include a needle connector coupled inside the needle housing and configured to support the laser guide member.

The needle unit may further include a needle holder coupled inside the needle housing and configured to support the needle member.

The needle RF laser treatment system may further include a needle driving unit provided inside the main body and configured to move the needle unit.

The needle driving unit may include: a driving motor coupled to the main body; a fixed body coupled to the main body; and a movable body having a first side connected to the driving motor to move in a longitudinal direction of the main body and a second side coupled to a lens unit configured to focus the laser, wherein the lens unit may be coupled to the needle unit.

The laser guide member may be an optical fiber.

The needle RF laser treatment system may further include a laser supply cable coupled to the main body and configured to supply laser to the laser guide member.

The needle RF laser treatment system may further include a lens unit having a first side coupled to the needle unit and a second side coupled to the laser supply cable, and configured to guide laser supplied from the laser supply cable to the laser guide member.

The lens unit may include a lens body having a first side coupled to the needle unit and a second side to which the laser supply cable is coupled, and a first side of a needle connector of the needle unit may be inserted and coupled in the lens body.

The lens unit may further include a lens member provided in the lens body.

The lens member may be a protection lens configured to protect the laser guide member from laser.

The lens member may be a focusing lens configured to focus laser supplied from the laser supply cable to the laser guide member.

The needle member may be provided in a number ranging from 4 to 128.

The laser guide member may be disposed up to a tip region of the needle member.

A polishing region may be provided at a front surface of the laser guide member.

Further, according to another aspect of the present disclosure, there may be provided a method of manufacturing a needle unit that is applied to a needle RF laser treatment system implemented by coupling a laser guide member configured to guide laser to a needle member configured to penetrate the skin of a user.

A first side of the laser guide member may be disposed in a tip region of the needle member.

The method may include a step of providing a polishing region by polishing a front surface of the laser guide member.

According to embodiments of the present disclosure, since laser energy is uniformly distributed to a plurality of needle members through a laser guide member, damage to the skin surface is significantly reduced during a treatment process. This leads to a reduction in a patient's pain and faster recovery.

Further, by delivering laser through the laser guide member, energy loss can be minimized and energy can be efficiently concentrated on a target area. Accordingly, the therapeutic effect is maximized.

Furthermore, since a plurality of needle members can expand a treatment area, treatment over a wider range than existing devices is possible, and accordingly, this can be utilized for treating various skin diseases.

In addition, since laser energy is delivered simultaneously to multiple needle members, the treatment process is more quickly performed. This provides time efficiency to both medical staff and patients.

Further, since energy intensity and distribution method can be adjusted through a combination of an optical fiber and a needle member, customized treatment can be performed in accordance with the condition of patients and treatment purposes.

### BRIEF DESCRIPTION OF THE DRAWINGS

FIG. 1 is a view schematically illustrating a needle RF laser treatment system according to an embodiment of the present disclosure;

FIG. 2 is a perspective view illustrating a state in which a tip housing shown in FIG. 1 is separated;

FIG. 3 is a perspective view illustrating a state in which a first fastening ring and an upper base body shown in FIG. 2 are separated;

FIG. 4 is a schematic right side view of the region of a lower base body shown in FIG. 3;

FIG. 5 is a perspective view illustrating a state in which a tip inner housing shown in FIG. 3 is separated;

FIG. 6 is a view schematically illustrating main parts of the embodiment;

FIG. 7 is a perspective view illustrating a state in which an LED PCB and a needle housing shown in FIG. 6 are separated;

FIG. 8 is a rear perspective view of FIG. 7 illustrating a state in which the LED PCB and the needle housing are removed;

FIG. 9 is a perspective view schematically illustrating the region of a needle connector shown in FIG. 7, a lens unit coupled to the needle connector, and a laser supply cable coupled to the lens unit;

FIG. 10 is a perspective view illustrating the needle unit shown in FIG. 5;

FIG. 11 is a perspective view illustrating main parts of the needle unit;

FIG. 12 is a cross-sectional view taken along line A-A′ shown in FIG. 10;

FIG. 13 is an exploded perspective view of FIG. 11;

FIG. 14 is an exploded perspective view of FIG. 10;

FIG. 15 is a view schematically illustrating a state in which a movable body is separated from a fixed body shown in FIG. 8;

FIG. 16 is an exploded perspective view schematically illustrating the region of the lens unit of the embodiment;

FIG. 17 is a view schematically illustrating main parts of the embodiment;

FIG. 18 is a view schematically illustrating a state in which a lens member protecting a laser guide member is provided in a lens body shown in FIG. 17; and

FIG. 19 is a view schematically illustrating a state in which a lens member focusing a laser to the laser guide member is provided in the lens body shown in FIG. 17.

### DETAILED DESCRIPTION

It is required to refer to the accompanying drawings exemplifying preferred embodiments of the present disclosure and the contents in the accompanying drawings to help sufficiently understand the present disclosure, the operational advantages of the present disclosure, and the objectives that are achieved by implementing the present disclosure.

The present disclosure will be described hereafter in detail by describing exemplary embodiments of the present disclosure with reference to the accompanying drawings. Like reference numerals given in the drawings indicate like components.

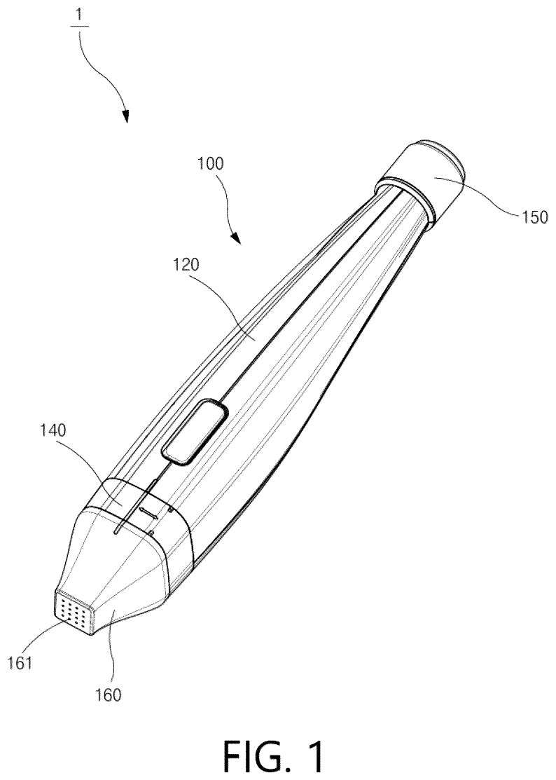

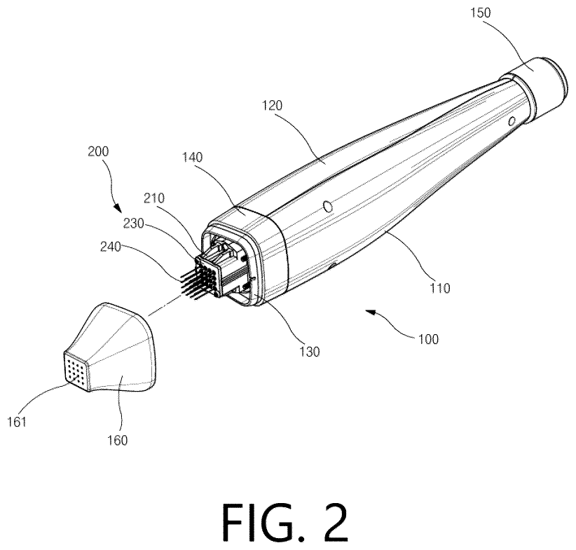

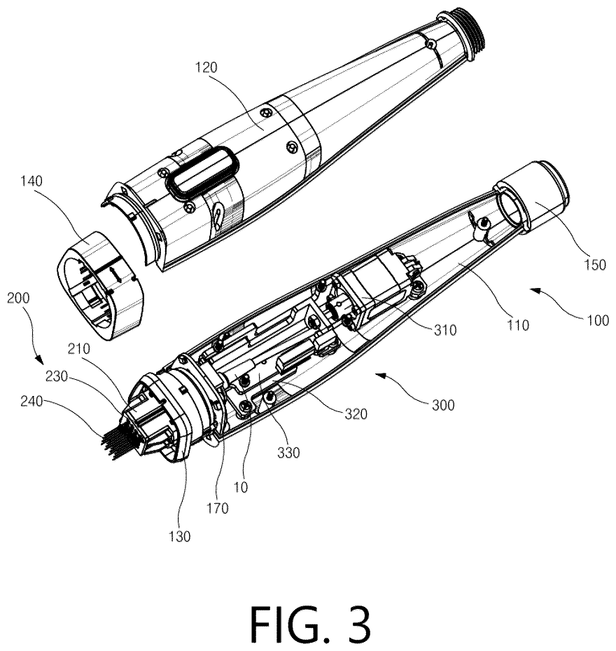

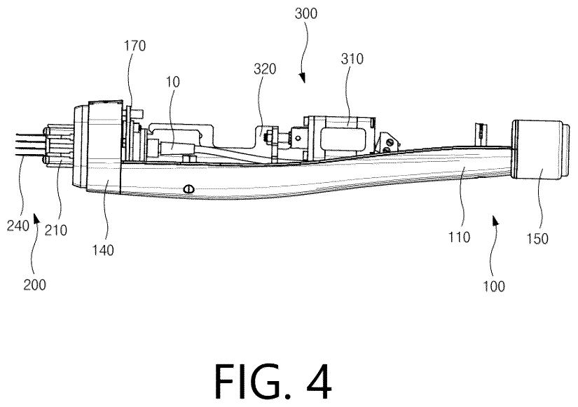

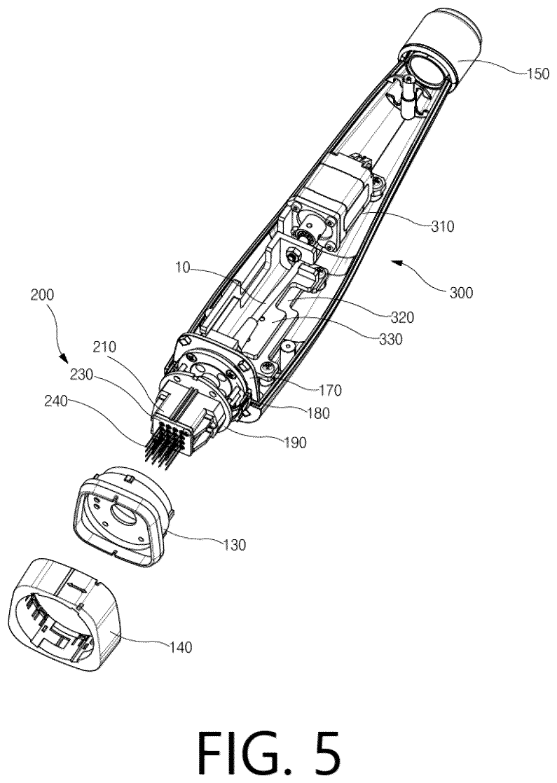

FIG. 1 is a view schematically illustrating a needle RF laser treatment system according to an embodiment of the present disclosure, FIG. 2 is a perspective view illustrating a state in which a tip housing shown in FIG. 1 is separated, FIG. 3 is a perspective view illustrating a state in which a first fastening ring and an upper base body shown in FIG. 2 are separated, FIG. 4 is a schematic right side view of the region of a lower base body shown in FIG. 3, and FIG. 5 is a perspective view illustrating a state in which a tip inner housing shown in FIG. 3 is separated.

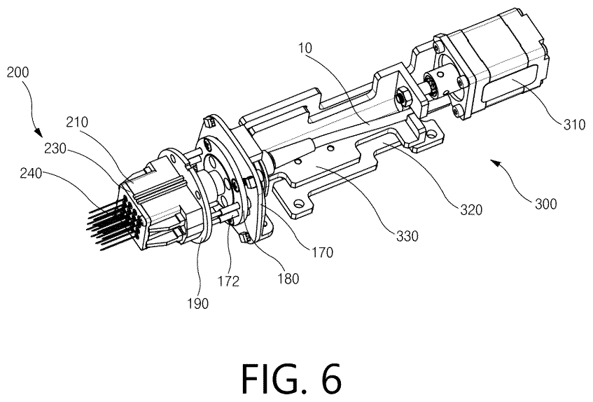

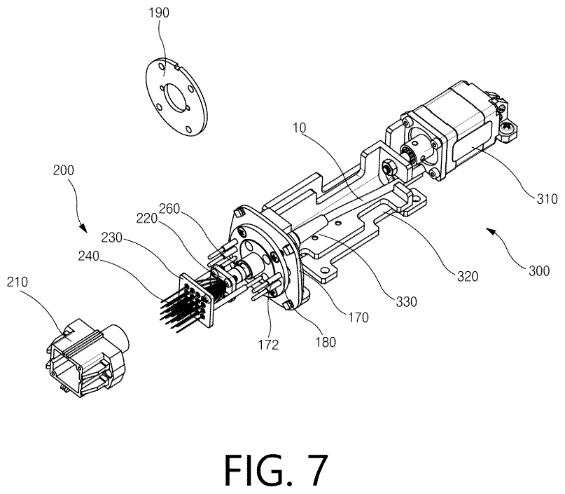

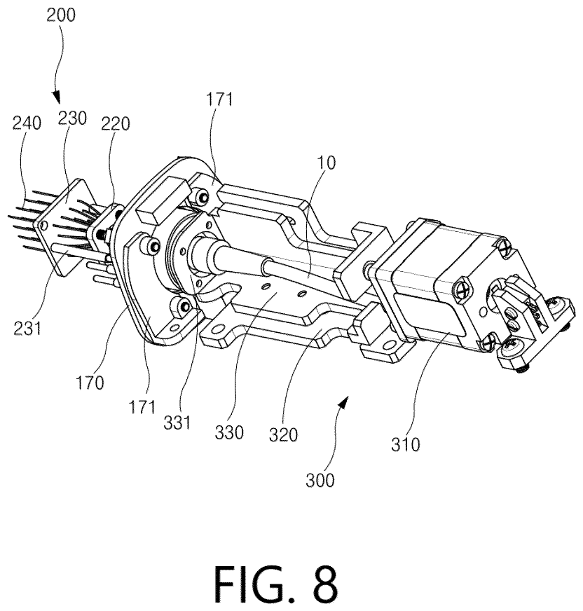

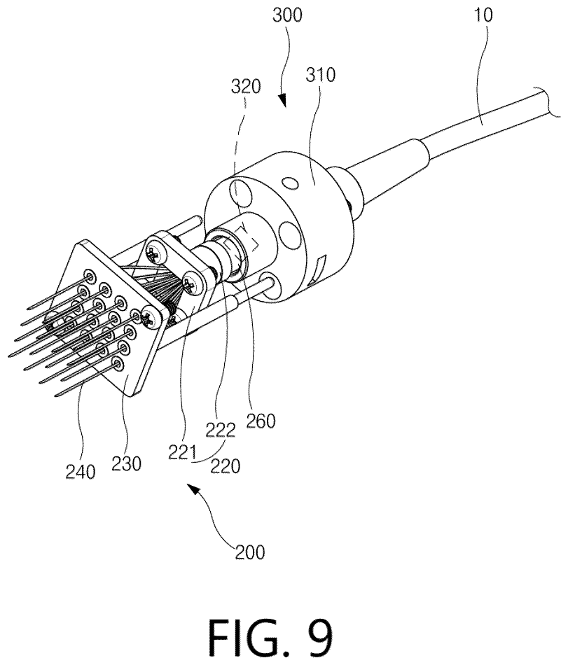

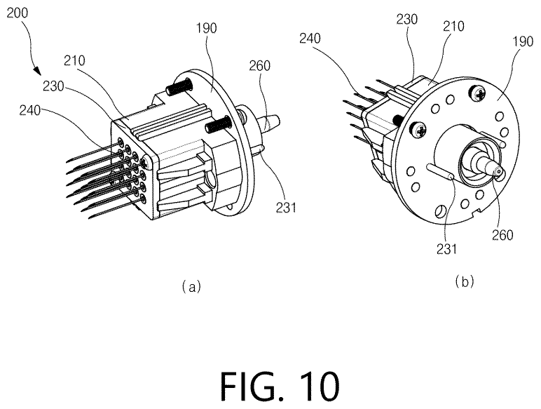

Further, FIG. 6 is a view schematically illustrating main parts of the embodiment, FIG. 7 is a perspective view illustrating a state in which an LED PCB and a needle housing shown in FIG. 6 are separated, FIG. 8 is a rear perspective view of FIG. 7 illustrating a state in which the LED PCB and the needle housing are removed, FIG. 9 is a perspective view schematically illustrating the region of a needle connector shown in FIG. 7, a lens unit coupled to the needle connector, and a laser supply cable coupled to the lens unit, and FIG. 10 is a perspective view illustrating the needle unit shown in FIG. 5.

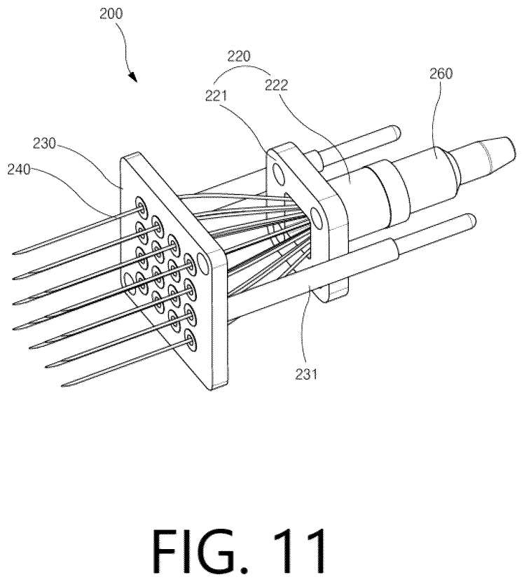

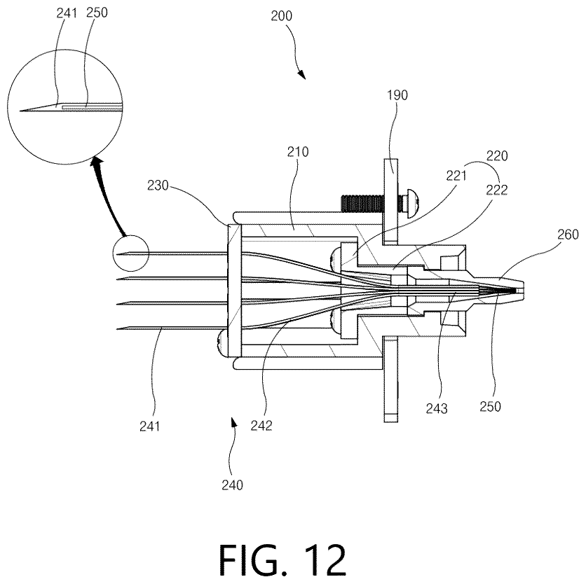

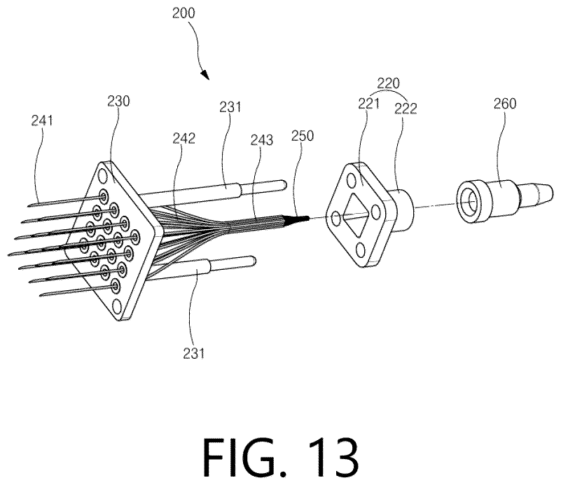

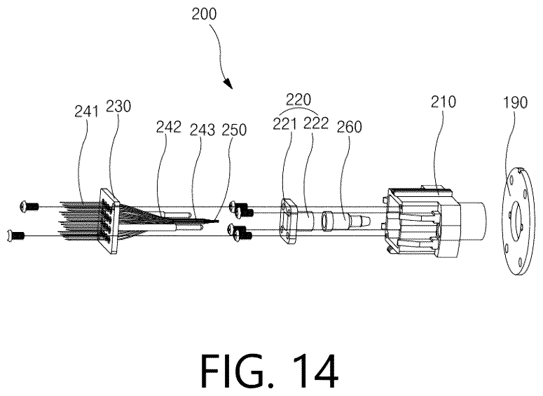

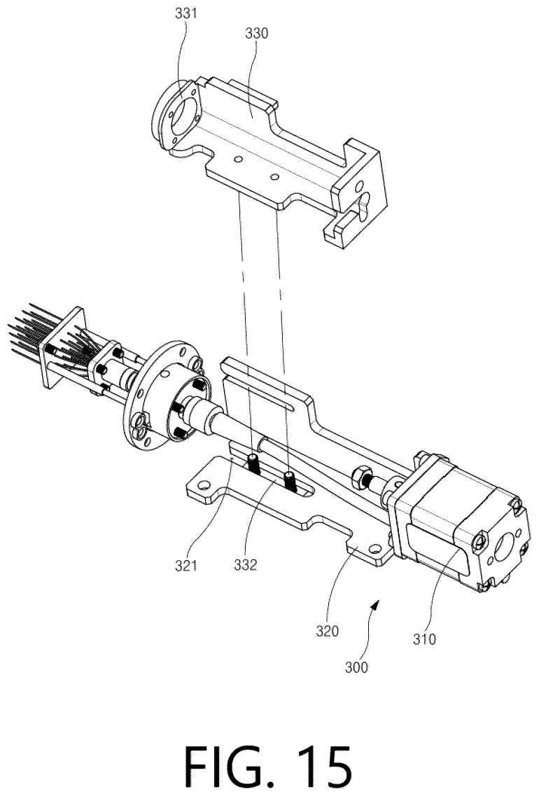

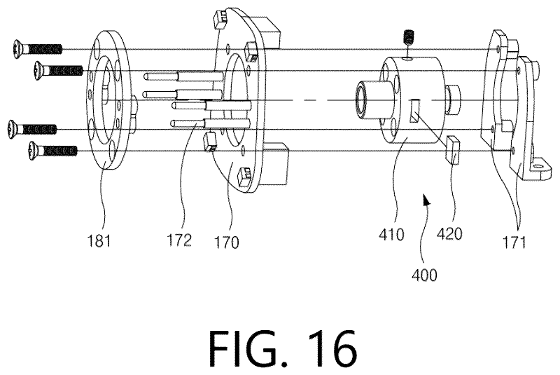

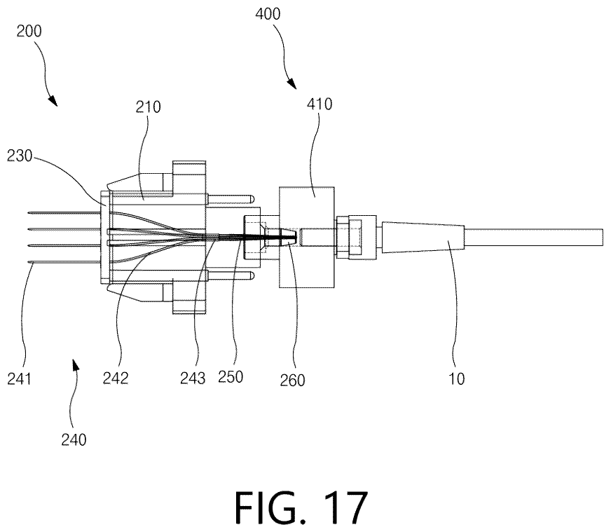

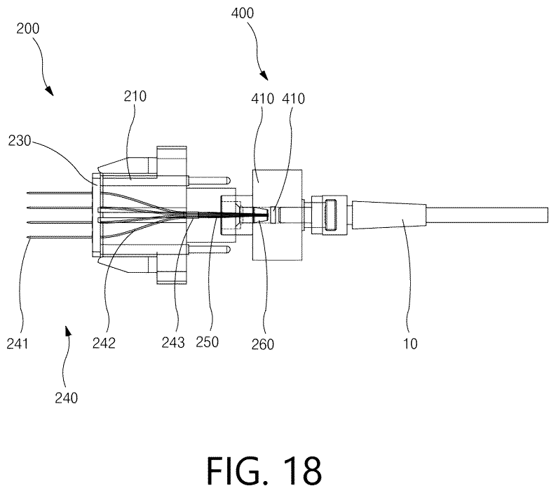

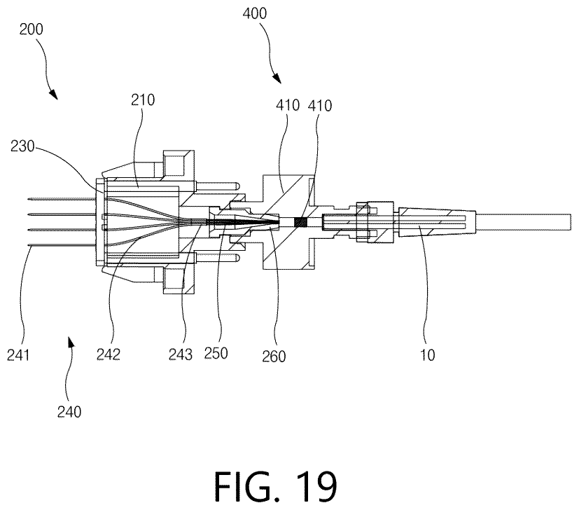

Further, FIG. 11 is a perspective view illustrating main parts of the needle unit, FIG. 12 is a cross-sectional view taken along line A-A′ shown in FIG. 10, FIG. 13 is an exploded perspective view of FIG. 11, FIG. 14 is an exploded perspective view of FIG. 10, FIG. 15 is a view schematically illustrating a state in which a movable body is separated from a fixed body shown in FIG. 8, FIG. 16 is an exploded perspective view schematically illustrating the region of the lens unit of the embodiment, FIG. 17 is a view schematically illustrating main parts of the embodiment, FIG. 18 is a view schematically illustrating a state in which a lens member protecting a laser guide member is provided in a lens body shown in FIG. 17, and FIG. 19 is a view schematically illustrating a state in which a lens member focusing a laser to the laser guide member is provided in the lens body shown in FIG. 17.

As illustrated in these figures, a needle RF laser treatment system 1 according to this embodiment includes: a main body 100; a needle unit 200 movably provided in the main body 100, penetrating the skin of a user, and emitting laser; a needle driving unit 300 provided in the main body 100 and moving the needle unit 200; and a lens unit 400 provided in the main body 100 and guiding laser supplied through a laser supply cable 10 to the needle unit 200.

The main body 100, as illustrated in FIG. 3, includes: a lower base body 110; an upper base body 120 disposed over the lower base body 110; a tip inner housing 130 coupled to a front region where the lower base body 110 and the upper base body 120 are coupled; a first fastening ring 140 coupled to the front region of the lower base body 110 and the upper base body 120; a second fastening ring 150 coupled to the rear region of the lower base body 110 and the upper base body 120; a tip housing 160 detachably coupled to the tip inner housing 130; an LED PCB 170 coupled to the front region of the lower base body 110 and the upper base body 120; a front cover 180 coupled to the front of the LED PCB 170; and a tip PCB 190 coupled to the rear of a needle housing 210.

In this embodiment, as illustrated in FIG. 2, a plurality of housing holes 161 may be spaced apart in the tip housing 160. In this embodiment, a plurality of needle members 240 may protrude through the plurality of housing holes 161 and penetrate the skin of a user.

In this embodiment, as illustrated in FIG. 8, a support cover 171 may be coupled to a rear wall of the LED PCB 170, and a lower portion of the support cover 171 may be detachably coupled to the lower base body 110, thereby supporting the LED PCB 170. In this embodiment, the top of the LED PCB 170 may be supported by the upper base body 120. In this embodiment, as illustrated in FIG. 16, a plurality of PCB pins 172 may be provided on the front wall of the PCB 170. In this embodiment, as illustrated in FIG. 6, the plurality of PCB pins 172 can pass through the front cover 180 and then be coupled to the tip PCB 190.

The needle unit 200 is provided in the main body 100, is moved by the needle driving unit 300, and can emit laser, for example RF laser, into the skin of a user.

In this embodiment, the needle unit 200 includes: a needle housing 210 coupled to the tip PCB 190, as illustrated in FIG. 6; a needle holder 220 coupled inside the needle housing 210, as illustrated in FIG. 12, and holding a plurality of needle members 240; a needle base 230 disposed at the front portion in the needle housing 210 and through which the plurality of needle members 240 protrudes forward; the plurality of needle members 240 supported at first sides in the holes provided in the needle base 230, penetrating the skin of a user, and emitting laser; a plurality of laser guide members 250 coupled to rears of the plurality of needle members 240 and supplying laser to the plurality of needle members 240, respectively; and a needle connector 260 having a first side coupled to the needle holder 220 and a second side coupled to a lens body 410 of the lens unit 400, and fixing the plurality of laser guide members 250.

In this embodiment, the needle holder 220, as illustrated in FIG. 11, includes a holder base 221 and a holder body 222 provided at the rear portion of the holder base 221 and to which the needle connector 260 is coupled. In this embodiment, the holder base 221, as illustrated in FIG. 12, may be detachably coupled inside needle housing 210 using a plurality of screw bolts.

In this embodiment, as illustrated in FIG. 11, a plurality of needle pins 231 is provided at the rear portion of the needle base 230, and the plurality of needle pins 231 may pass through the needle housing 210 and be coupled to the tip PCB 190, as illustrated in FIG. 10(b).

In this embodiment, as illustrated in FIG. 12, the needle member 240 includes: a first region 241 having a first end protruding outward from the needle base 230, penetrating the skin of a user, and disposed parallel to the propagation direction of laser; a second region 242 connected to a second end of the first region 241 and obliquely disposed inside the needle housing 210; and a third region 243 disposed inside the needle unit 200, having a first end connected to the second region 242, and having a second connected to the laser supply cable 10 that supplies laser. In this embodiment, the second region 242 of the needle member 240, as illustrated in FIG. 12, may be supported by the needle holder 220. In this embodiment, most of the third region 243, as illustrated in FIG. 12, except for a portion at the front side, may be disposed inside the needle connector 260. In this embodiment, the needle member 240 may be replaced with a member having a function of repeatedly penetrating the skin of a user and supplying laser to the skin. In this embodiment, a plurality of needle members 240 may be provided. In this embodiment, the needle members 240 may be provided in a number ranging from 4 to 128.

In this embodiment, as illustrated in FIG. 12, the laser guide members 250 are coupled to ends of the needle members 240, that is, to the third regions 243, so they can supply laser supplied from the laser supply cable 10 to the plurality of needle members 240. In this embodiment, the laser guide member 250 may be an optical fiber, and is not limited thereto but may be replaced with a known element for guiding laser. In this embodiment, as illustrated in FIG. 12, the tip of the laser guide member 250 may be disposed up to the tip region of the needle member 240. In this embodiment, the laser guide member 250 may be provided at the tip of the needle member 240 not to be exposed through an open hole formed at the tip of the needle member 240. In this embodiment, a polishing region may be provided at the front surface of the laser guide member 250 so that laser emitted from the laser guide member 250 can be directed to the skin of a user with minimum loss. In this embodiment, the polishing region may be a foremost region of the laser guide member 250 that appears perpendicular when FIG. 12 is viewed from left to right.

In this embodiment, as illustrated in FIG. 12, the needle connector 260 has a first side coupled to the holder body 222 of the needle holder 220 and a second side coupled to the lens unit 400, so that, when the needle driving unit 300 operates, the needle unit 200 can be moved together with the lens unit 400.

The needle driving unit 300, as illustrated in FIG. 3, is coupled to the lower base body 110 of the main body 100 and can reciprocate the lens unit 400 and the needle unit 200 toward the skin of a user.

In this embodiment, as illustrated in FIG. 5, the needle driving unit 300 includes: a driving motor 310 coupled to the lower base body 110; a fixed body 320 coupled to the lower base body 110; and a movable body 330 having a first side connected to the driving motor 310 to move in the longitudinal direction of the main body 100 and a second side coupled to the lens unit 400 that focuses laser.

In this embodiment, a motor shaft of the driving motor 310 may be detachably coupled to a rear of the movable body 330 using nuts after being provided at the rear portion. In this embodiment, the driving motor 310 may be a stepping motor.

In this embodiment, as illustrated in FIG. 15, a guide hole 321 may be provided at a front of the fixed body 320, and a guide rail 332 coupled to a bottom surface of the movable body 330 may be supported under the guide hole 321. As a result, the movable body 3300 can be stably moved in the longitudinal direction of the main body 100.

The lens unit 400, as illustrated in FIG. 15, is detachably coupled to a coupling plate 331 of the movable body 330 using screws and can supply laser supplied from the laser supply cable 10 to the plurality of laser guide members 250.

In this embodiment, as illustrated in FIG. 16, the lens unit 400 includes a lens body 410 and a lens member 420 coupled inside the lens body 410 and guiding laser.

In this embodiment, the needle connector 260 of the needle unit 200 may be fitted to a front of the lens body 410, and the laser supply cable 10 may be coupled to a rear of the lens body 410.

In the embodiment, as illustrated in FIG. 17, the lens unit 400 may be used with only the lens body without the lens member 420. In this case, laser supplied from the laser supply cable 10 can be supplied into the needle connector 260 through the inside of the lens body 410.

In this embodiment, as illustrated in FIG. 18, the lens member 420 may be a protection lens and it can protect the laser guide members 250 from laser.

In this embodiment, as illustrated in FIG. 19, the lens member 420 may be a focusing lens and it can focus laser supplied from the laser supply cable 10 to the laser guide members 250.

Meanwhile, a method for manufacturing a needle unit that is applied to the needle RF laser treatment system according to this embodiment may be implemented by coupling the laser guide members 250, which guide laser, to the needle members 240 that penetrate the skin of a user.

In this embodiment, a first side of the laser guide member 250 may be fitted to the rear end of the needle member 240, for example, to the third region 243.

In this embodiment, the first side of the laser guide member 250 may be disposed at the tip region of the needle member 240. In this embodiment, the tip of the laser guide member 250 may be provided in the tip region of the needle member 240 not to be exposed through the open hole formed at the tip of the needle member 240.

In this embodiment, a polishing region may be provided at the front surface of the laser guide member 250 so that laser emitted from the laser guide member 250 can be directed to the skin of a user with minimum loss.

The present disclosure is not limited to the described embodiments, and it will be apparent to those skilled in the art that various modifications and alterations can be made without departing from the spirit and scope of the present disclosure. Accordingly, such modifications or alterations should be construed as falling within the claims of the present disclosure.

### EXPLANATION OF REFERENCE NUMERALS

1: Needle RF Laser treatment system
100: Main body
110: Lower base body
120: Upper base body
130: Tip inner housing
140: First fastening ring
150: Second fastening ring
160: Tip housing
161: Housing hole
170: LED PCB
171: Support cover
172: PCB pin
180: Front cover
190: Tip PCB
200: Needle unit
210: Needle housing
220: Needle holder
221: Holder base
222: Holder body
230: Needle base
231: Needle pin
240: Needle member
241: First region
242: Second region
243: Third region
250: Laser guide member
260: Needle connector
300: Needle driving unit
310: Driving motor
320: Fixed body
321: Guide hole
330: Movable body
331: Coupling plate
332: Guide rail
400: Lens unit
410: Lens body
420: Lens member
10: Laser supply cable

What is claimed is:

1. A needle RF laser treatment system having a needle unit including:
a needle member provided in a main body and configured to penetrate the skin of a user; and
a laser guide member coupled to the needle member and configured to supply laser to the needle member.

2. The needle RF laser treatment system of claim 1, wherein the needle member includes a plurality needle members, and the laser guide member is coupled to each of the plurality of needle members to supply laser to each of the needle members.

3. The needle RF laser treatment system of claim 1, wherein the needle member includes:
a first region having a first end protruding outward from the needle unit, penetrating the skin of a user, and disposed parallel to a propagation direction of laser;
a second region connected to a second end of the first region and obliquely disposed inside the needle unit; and
a third region disposed inside the needle unit, having a first end connected to the second region 242, and having a second end connected to a laser supply cable configured to supply laser.

4. The needle RF laser treatment system of claim 1, wherein the needle unit includes:
a needle housing coupled to the main body; and
a needle base coupled to the needle housing and configured to support the needle member exposed outside the needle housing.

5. The needle RF laser treatment system of claim 4, wherein the needle unit further includes a needle connector coupled inside the needle housing and configured to support the laser guide member.

6. The needle RF laser treatment system of claim 4, wherein the needle unit further includes a needle holder coupled inside the needle housing and configured to support the needle member.

7. The needle RF laser treatment system of claim 1, further comprising a needle driving unit provided inside the main body and configured to move the needle unit.

8. The needle RF laser treatment system of claim 7, wherein the needle driving unit includes:
a driving motor coupled to the main body;
a fixed body coupled to the main body; and
a movable body having a first side connected to the driving motor to move in a longitudinal direction of the main body and a second side coupled to a lens unit configured to focus the laser,
wherein the lens unit is coupled to the needle unit.

9. The needle RF laser treatment system of claim 1, wherein the laser guide member is an optical fiber.

10. The needle RF laser treatment system of claim 1, further comprising a laser supply cable coupled to the main body and configured to supply laser to the laser guide member.

11. The needle RF laser treatment system of claim 10, further comprising a lens unit having a first side coupled to the needle unit and a second side coupled to the laser supply cable, and configured to guide laser supplied from the laser supply cable to the laser guide member.

12. The needle RF laser treatment system of claim 11, wherein the lens unit includes a lens body having a first side coupled to the needle unit and a second side to which the laser supply cable is coupled, and
a first side of a needle connector of the needle unit is inserted and coupled in the lens body.

13. The needle RF laser treatment system of claim 12, wherein the lens unit further includes a lens member provided in the lens body.

14. The needle RF laser treatment system of claim 13, wherein the lens member is a protection lens configured to protect the laser guide member from laser.

15. The needle RF laser treatment system of claim 13, wherein the lens member is a focusing lens configured to focus laser supplied from the laser supply cable to the laser guide member.

16. The needle RF laser treatment system of claim 1, wherein the needle member is provided in a number ranging from 4 to 128.

17. The needle RF laser treatment system of claim 1, wherein the laser guide member is disposed up to a tip region of the needle member.

18. The needle RF laser treatment system of claim 1, wherein a polishing region is provided at a front surface of the laser guide member.

19. A method of manufacturing a needle unit that is applied to a needle RF laser treatment system implemented by coupling a laser guide member configured to guide laser to a needle member configured to penetrate the skin of a user.

20. The method of claim 19, wherein a first side of the laser guide member is disposed in a tip region of the needle member.

21. The method of claim 19, comprising a step of providing a polishing region by polishing a front surface of the laser guide member.
